(19) United States
(12) Patent Application Publication
(10) Pub. No.: US 2023/0210545 A1
(43) Pub. Date: Jul. 6, 2023

(54) EXPANDABLE INTRALUMINAL DEVICE

(71) Applicant: Deepin Technologies, LLC., San Jose, CA (US)

(72) Inventors: CHRIS ZALER, Los Gatos, CA (US); Tony Le, Sunnyvale, CA (US)

(73) Assignee: Deepin Technologies, LLC., San Jose, CA (US)

(21) Appl. No.: 18/072,768

(22) Filed: Dec. 1, 2022

Related U.S. Application Data
(60) Provisional application No. 63/295,340, filed on Dec. 30, 2021.

(51) Int. Cl. A61B 17/221

(52) U.S. Cl. CPC A61B 17/221; A61B 2017/00867

(57) ABSTRACT

An intraluminal device includes a radially expandable body and a control member operable to expand and contract the radially expandable body. The radially expandable body comprises plural sets of first filaments spiraling in a first direction from a proximal end portion to a distal end portion of the radially expandable body and plural sets of second filaments spiraling in a second direction from the proximal end portion to the distal end portion. The plural sets of first filaments intersect the plural sets of second filaments, forming plural cross sections of the radially expandable body. At least one of the plural sets of first filaments comprises a filament constructed of a radiopaque material.

### CROSS-REFERENCE TO RELATED APPLICATIONS

This application claims priority to U.S. provisional patent application No. 63/295,340 filed Dec. 30, 2021 entitled “Expandable Intraluminal Device,” the disclosure of which is hereby incorporated by reference in its entirety.

### TECHNICAL FIELD

This application relates generally to medical devices and methods of making and using medical devices. In particular, various embodiments of an expandable intraluminal device and a method of removing obstructions such as clots from human blood vessels are described.

### BACKGROUND

Intraluminal devices such as stent retrievers are known and commonly used to remove obstructions, such as clots, from human blood vessels. A stent retriever includes an expandable mesh-like structure, which can be in a thin compact form to allow it to penetrate through a blood clot. Once incapsulated in a clot, the mesh-like structure is expanded, exerting a radial force on the clot to expand into and capture the clot inside the mesh-like structure. The clot can be removed from the vessel by retrieving the stent retriever.

Conventional stent retrievers are constructed of shape memory materials and rely on the shape memory property to expand to a certain dimension. However, relying solely on the shape memory property does not allow the user to control the radial expansion of a stent retriever which may be needed for effective penetration through a blood clot and successful removal of the clot through variably sized vessels.

Therefore, there remains a need for improved intraluminal devices to treat various medical conditions in blood vessels or other hollow body organs. It would be desirable to provide an expandable intraluminal device that allows the user to control the expansion and contraction of the device, and thus the radial force of expansion, to facilitate effective penetration and removal of an obstruction from blood vessels.

### SUMMARY

In one aspect, embodiments of the disclosure feature an intraluminal device. In general, an embodiment of the intraluminal device comprises a radially expandable body having a proximal end portion and a distal end portion and a control member operable to expand and contract the radially expandable body. The radially expandable body comprises plural sets of first filaments spiraling in a first direction from the proximal end portion to the distal end portion and plural sets of second filaments spiraling in a second direction from the proximal end portion to the distal end portion. The plural sets of first filaments intersect the plural sets of second filaments, forming plural cross sections of the radially expandable body. At least one of the plural sets of first filaments comprises a filament constructed of a radiopaque material.

In various embodiments of the aspect, each of the plural sets of first filaments comprises a filament constructed of a radiopaque material.

In various embodiments of the aspect, each of the plural sets of second filaments is free of a filament constructed of a radiopaque material.

In various embodiments of the aspect, each of the plural sets of first filaments comprises two filaments that are twisted and each of the plural sets of the second filaments comprises two filaments that are twisted.

In various embodiments of the aspect, at a cross section where one of the plural sets of first filaments and one of the plural sets of second filaments intersect, one of the two filaments of the one of plural sets of first filaments passes between the two filaments of the one of plural sets of second filaments, and one of the two filaments of the one of plural sets of second filaments passes between the two filaments of the one of plural sets of first filaments. In a specific embodiment, the plural sets of first filaments each comprises a radiopaque filament and a non-radiopaque filament, and the plural sets of second filaments each comprises two non-radiopaque filaments.

In various embodiments of the aspect, the control member comprises a pull wire having a distal end portion coupled to the distal end portion of the radially expandable body, allowing to apply an axial force to move the distal and proximal end portions of the radially expandable body relative to each other.

In another aspect, embodiments of the disclosure feature an intraluminal device. The intraluminal device comprises a radially expandable body having a proximal end portion and a distal end portion and a control member operable to expand and contract the radially expandable body. The radially expandable body comprises plural sets of first filaments spiraling in a first direction from the proximal end portion to the distal end portion, plural sets of second filaments spiraling in a second direction from the proximal end portion to the distal end portion. The plural sets of second filaments intersect the plural sets of first filaments, forming plural cross sections of the radially expandable body. The intraluminal device further comprises plural radiopaque filaments including at least a first radiopaque filament and a second radiopaque filament. The first radiopaque filament comprises a first section grouped with one of the plural sets of first filaments, and the second radiopaque filament comprises a first section grouped with one of the plural sets of second filaments. The first radiopaque filament and the second radiopaque filament are twisted at a cross section where the one of the plural sets of first filaments and the one of the plural sets of second filaments intersect, to allow a second section of the first radiopaque filament to be grouped with the one of the plural sets of second filaments and allow a second section of the second radiopaque filament to be grouped with the one of the plural sets of first filaments.

In various embodiments of the aspect, each of the plural sets of first filaments comprises two filaments that are twisted, and each of the plural sets of second filaments comprises two filaments that are twisted.

In various embodiments of the aspect, the number of the plural radiopaque filaments is equal to the total number of sets of first and second filaments.

In a further aspect, embodiments of the disclosure feature an intraluminal device. The intraluminal device comprises a radially expandable body having a proximal end portion and a distal end portion and a control member operable to expand and contract the radially expandable body. The radially expandable body comprises plural elongate ribbons each extending from the proximal end portion to the distal end portion and one or more filaments coupled to the plural elongate ribbons forming a mesh structure.

In various embodiments of the aspect, the plural elongate ribbons are formed by cutting plural slits in a tubular member.

In various embodiments of the aspect, each of the plural ribbons comprises plural attachment features to couple the one or more filaments.

In various embodiments of the aspect, the plural attachment features are arranged to allow the one or more filaments to form plural circular filaments each intersecting the plural elongate ribbons. The plural circular filaments are generally perpendicular to a longitudinal axis of the radially expandable body.

In various embodiments of the aspect, the plural attachment features are arranged to allow the one or more filaments to extend spirally from the proximal end portion to the distal end portion and intersect the plural elongate ribbons.

In various embodiments of the aspect, the plural attachment features are arranged to allow the one or more filaments to sway between two or more elongate ribbons in a sine-wave manner.

In various embodiments of the aspect, the plural elongate ribbons are constructed of a shape memory material.

In various embodiments of the aspect, the plural elongate ribbons are generally evenly spaced apart circumferentially.

In various embodiments of the aspect, the plural elongate ribbons extend spirally from the proximal end portion to the distal end portion in a first direction, and the one or more plural filaments extend spirally from the proximal end portion to the distal end portion in a second direction opposite to the first direction. In a specific embodiment, the plural elongate ribbons are formed by cutting plural slits in a spiral pattern in a tubular member constructed of a shape memory material.

This Summary is provided to introduce selected aspects and embodiments of this disclosure in a simplified form and is not intended to identify key features or essential characteristics of the claimed subject matter, nor is it intended to be used as an aid in determining the scope of the claimed subject matter. The selected aspects and embodiments are presented merely to provide the reader with a brief summary of certain forms the invention might take and are not intended to limit the scope of the invention. Other aspects and embodiments of the disclosure are described in the section of Detailed Description.

These and various other aspects, embodiments, features, and advantages of the disclosure will become better understood upon reading of the following detailed description in conjunction with the accompanying drawings.

### BRIEF DESCRIPTION OF THE DRAWINGS

FIG. 1A depicts an example intraluminal device according to embodiments of the disclosure.

FIG. 1B depicts the example intraluminal device of FIG. 1A in a different view according to embodiments of the disclosure.

FIG. 2 depicts an example pattern of cross sections of an intraluminal device according to embodiments of the disclosure.

FIG. 3A depicts an example pattern of cross sections of an intraluminal device according to alternative embodiments of the disclosure. FIG. 3B is an enlarged view showing details of a cross section pattern shown in FIG. 3A.

FIG. 4 depicts an example device including plural elongate ribbons formed by cutting a material from a single tube according to embodiments of the disclosure.

FIGS. 5A-5B depict an example intraluminal device according to some embodiments of the disclosure.

FIG. 6 depicts an example intraluminal device according to alternative embodiments of the disclosure.

FIG. 7 depicts an example intraluminal device according to alternative embodiments of the disclosure.

FIG. 8A depicts an example device including plural elongate ribbons formed by cutting a material from a single tube according to alternative embodiments of the disclosure. FIG. 8B depicts the example device of FIG. 8A in a different view according to alternative embodiments of the disclosure.

### DETAILED DESCRIPTION OF EMBODIMENTS

With reference to the figures, various embodiments of an intraluminal device will now be described. It should be noted that the figures are intended for illustration of embodiments but not for exhaustive description or limitation on the scope of the disclosure. Alternative structures and components will be readily recognized as being viable without departing from the principle of the claimed invention.

Intraluminal Device Including a Spiral Wrap

Embodiments of the disclosure provide an intraluminal device which allows the user to control radial expansion and contraction of the device. The intraluminal device comprises a radially expandable body, or a mesh-like structure, or a continuous spiral wrap. Starting in a thin compact form, the intraluminal device can penetrate through an obstruction such as a clot in a blood vessel. Once the distal portion of the spiral wrap has traveled through a clot and the spiral wrap is incapsulated in the clot, the user can manually expand the spiral wrap. With manual expansion, the user can increase and/or decrease the radial force to help the spiral wrap work its way through the clot. Providing a capability of control of radial expansion of the spiral wrap allows the user to achieve greater penetration of a clot as compared to conventional stent retrievers. The user can expand the spiral wrap more for a larger vessel or less for a smaller vessel. During expansion, the spiral wrap can penetrate radially through the clot and create a hold on the bulk of the clot. The more the spiral wrap expands, the better hold the device has on the clot. Once the spiral wrap has fully expanded, the user can then remove the clot from the vessel by retrieving the spiral wrap. Greater hold on the clot gives greater confidence that the entire clot will be extracted during the first retrieval of the spiral wrap from the patient. Conventional stent retriever relies on the shape memory property of the stent material to expand. Relying solely on shape memory property limits the amount of radial force a stent retriever can achieve and does not allow the user to control of the radial expansion which may be needed for effective penetration and successful removal of a clot through variably sized vessels.

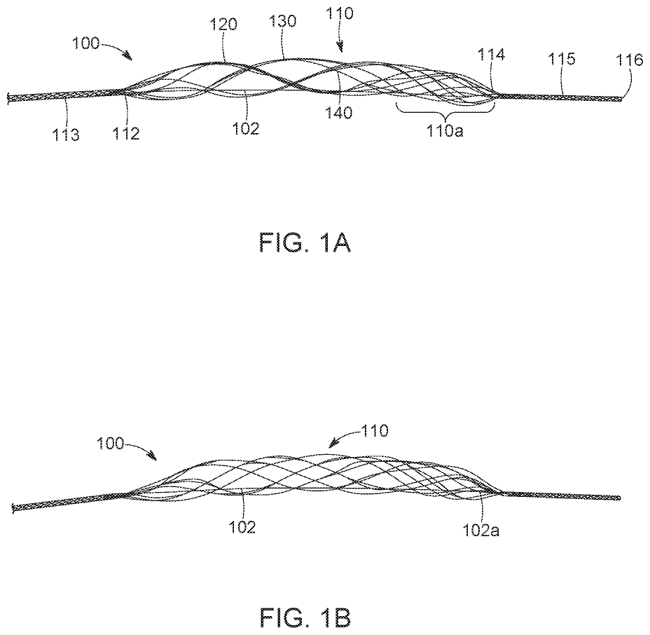

FIGS. 1A-1B depict an example intraluminal device 100 according to embodiments of the disclosure. In general, the intraluminal device 100 comprises a radially expandable body 110 and a control member 102 operable to control radial expansion and contraction of the radially expandable body 110. The radially expandable body 110 comprises plural sets of first filaments 120 extending spirally in a first direction e.g., clockwise, from a proximal end portion 112 to a distal end portion 114 of the radially expandable body 110. The radially expandable body 110 further comprises plural sets of second filaments 130 extending spirally in a second direction e.g., counterclockwise, from the proximal end portion 112 to the distal end portion 114 of the radially expandable body 110. The plural sets of first filaments 120 intersect the plural sets of second filaments 130, forming plural cross sections 140 and defining plural openings or cells of the radially expandable body 110. In the disclosure, the term “radially expandable body” may be used interchangeably with the terms “mesh,” “mesh body,” “mesh-like structure,” and “spiral wrap.” The control member 102 comprises a distal end portion 102a coupled to the distal end portion 114 of the radially expandable body 110. The proximal end portion of the control member 102 may be coupled to an operating device such as a handle (not shown in FIGS. 1A-1B). The operating device may apply an axial force to the control member 102, causing the distal and proximal end portions 112, 114 of the radially expandable body 110 to move relative to each other to expand and contract the radially expandable body 110. By way of example, the control member 102 may be in the form of a pull or push wire, configured to pull or push the distal end portion 114 of the radially expandable body 110 and thus cause the distal and proximal end portions 112, 114 of the radially expandable body 110 to move relative to each other, allowing the outer diameter of the expandable body 110 to increase or decrease. The control member 102 allows the user to control the outer diameter of the expandable body 102 and manipulate the radial force of expansion to aid in penetration and removal of an obstruction such as a blood clot.

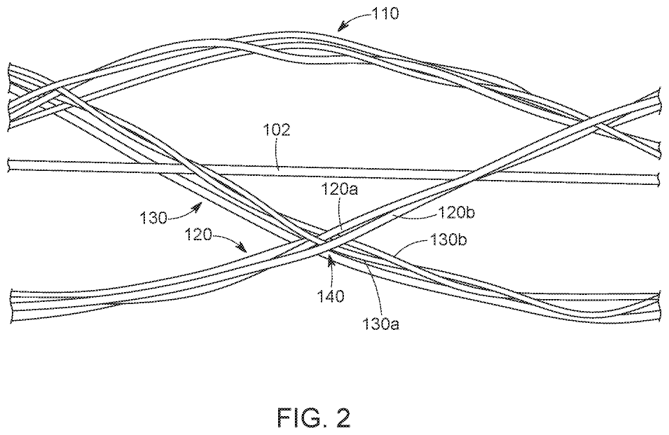

With reference to FIG. 2, according to embodiments of the disclosure, a set of first filaments 120 and a set of second filaments 130 may each include two or more filaments that are twisted. Alternatively, the filaments of a set are untwisted. At a cross section 140 where a set of first filaments 120 and a set of second filaments 130 intersect, one filament 120a of a set of first filaments 120 may pass between two filaments 130a, 130b of a set of second filaments 130. Alternatively, or additionally, one filament 130a of a set of second filaments 130 may pass between two filaments 120a, 120b of a set of first filaments 120. As such, the set of first filaments 120 may rotate about a filament 130a of the set of second filaments 130, and/or, the set of second filaments 130 may rotate about a filament 120a of the set of first filaments 120, thereby allowing the mesh body 110 to freely expand to variable diameters. The twist between first filaments 120a, 120b of a set 120 prevents or limits slipping of the filament 130a of a set of second filaments 130, and/or, the twist between second filaments 130a, 130b of a set 130 prevents or limits slipping of the filament 120a of a set of first filaments 120, allowing the cells of the mesh body 110 to maintain a predetermined size during expansion and contraction. Therefore, the pattern or configuration of cross sections 140 of the first and second filaments 120, 130 according to embodiments of the disclosure allows the mesh body 110 to expand to variable diameters while maintaining a predetermined cell size. This feature of the intraluminal device 100 of the disclosure helps create a precise, repeatable expansion and radial force. Free and precise expansion of the mesh body 110 allows for ease of application of radial force and creates tactile feedback to the user. It should be noted that while a set of first or second filaments 120, 130 is shown in FIG. 2 to include two filaments for purpose of illustration, in alternative embodiments, a set of first and/or second filaments 120, 130 may include three, four, or more filaments respectively. In general, a set of first or second filaments may include 2-4 filaments.

With reference to FIG. 2, the radially expandable body may include one or more radiopaque filaments or filaments that are constructed of a radiopaque material. Radiopaque filaments are visible via fluoroscopy, aiding the user in operating the intraluminal device. By way of example, one of the plural sets of first filaments 120 may include a filament 120b constructed of a radiopaque material. In some embodiments of the disclosure, each of the plural sets of first filaments 120 may include a filament constructed of a radiopaque material. In some embodiments, at least one of the plural sets of first filaments 120 may include a filament constructed of a radiopaque material whereas each of the plural sets of second filaments 130 is free of a radiopaque filament. In some embodiments, each of the plural sets of first filaments 120 may include a filament constructed of a radiopaque material whereas each of the plural sets of second filaments 130 is free of a radiopaque filament. Whereas a radiopaque filament allows the user to view the intraluminal device in the patient body via fluoroscopy, filaments constructed of a non-radiopaque material e.g., nitinol, can provide the device with greater structural strength, higher radial force, better twist resistance, or other desirable characteristics.

Radiopaque filaments can be constructed of a material comprising platinum, gold, tantalum, barium, lodin, bismuth, etc., or an alloy containing any of the above metals. In some embodiments, radiopaque filaments may be in the form of drawn filled tubing (DFT) wires, which may include a core of a radiopaque material such as platinum, gold, tantalum etc. and an outer sheath of a non-radiopaque material such as nitinol. Whereas the core of DFT wires provides radiopacity, the outer sheath of DFT wires can provide strength, flexibility, elasticity, and other desirable properties. Non-radiopaque filaments may be constructed of a material comprising nickel, titanium, nickel-titanium alloy such as nitinol, and other suitable materials. Nitinol exhibits flexibility, elasticity, shape memory and other desirable properties, and is a preferred material for constructing non-radiopaque filaments.

For illustration and by way of example, in a non-limiting specific embodiment of the disclosure, an intraluminal device may include 12 filaments, of which 9 are constructed of a non-radiopaque material and the remaining 3constructed of a radiopaque material. The 12 filaments may be grouped in 6 sets or pairs wherein each set or pair contains two filaments twisted or bundled together in extending from the proximal end portion to the distal end portion of the radially expandable body. Of the 6 sets or pairs of filaments, 3 sets extend spirally in a clockwise direction and 3 sets in a counterclockwise direction. Of the 3 sets of filaments that extend spirally in the same direction, each set contains a radiopaque filament and a non-radiopaque filament. At a cross section of the mesh body, a filament of a set of passes between two filaments of another set. As such, a set of two filaments may rotate about a filament of another set, thereby allowing the mesh body to freely expand. The twist between filaments of a set prevents or limits slipping of a filament of another set, thereby allowing the cell of the mesh body to maintain a predetermined size during expansion and contraction of the mesh body.

According to embodiments of the disclosure, the filaments may have a diameter ranging e.g., from 0.003″ to 0.006″ depending on applications. The mesh body may have a minimal diameter when in a thin compact form e.g., about 0.012″, and a maximal diameter when in a large expanded configuration e.g., about 0.019″. it should be noted that the dimensions are provided for understanding of the disclosure and the present claims are not so limited. The expansion of the mesh body can be controlled by operating the control member, allowing the mesh structure to have a diameter ranging from the minimal diameter and the maximal diameter, to adapt to variably sized blood vessels and provide variable radial expansion force for penetration and removal of a clot.

Returning to FIGS. 1A-1B, the plural sets of first filaments 120 and the plural sets of second filaments 130 may be joined at the proximal end 112 to form a multifilar coil 113. The flexible multifilar coil allows access of the mesh in a thin compact form through challenging vessels, e.g., tight curves or loops when navigating to a specific target location. At the distal end portion 114, the plural sets of first filaments 120 and the plural sets of second filaments 130 may also be joined to form a multifilar coil 115 and provided with an atraumatic tip 116. The atraumatic tip 116 can create a small leading edge to help positioning of the spiral wrap in the blood clot. The atraumatic tip 116 may be flexible to reduce the risk of damaging the blood vessel. The atraumatic tip 116 can be made of a radiopaque material such as platinum or the like to assist the user to view the intraluminal device 100 via fluoroscopy.

While not shown in FIGS. 1A-1B, the intraluminal device 100 of the disclosure may also include an operating device such as a handle to control and hold the mesh structure 110 in various expanded configurations. The proximal end of the control member 120 may be coupled to the operating device. By way of example, the operating device or handle may include a thumb slider that can slide and lock into different locations, allowing for variable expansion of the mesh structure 110 and holding an expanded configuration of the mesh structure 110 when needed. The control member 102 may comprise a wire of stainless steel, which can be spring tempered, or any other suitable materials. The control member 102 may be coupled to the distal end portion 114 of the expandable body 110, extends within the mesh structure 110, passes through the proximal end portion 112, and be coupled to the operating device. Alternatively, the control member 102 may be arranged outside of the mesh structure 110. The control member 102 may be configured to apply an axial force to the distal end portion 114 of the expandable body 110 e.g., a pulling force causing the expandable body 110 to radially expand, or a pushing force causing the expandable body 110 to radially contract.

With reference to FIGS. 1A-1B, according to embodiments of the disclosure, the expandable body 110 may include a portion 110a having a tighter weave pattern. The tighter weaved portion 110a is proximal to the distal end portion 114 of the expandable body 110 and can act as a backstop when removing a clot. A tight weave pattern creates smaller cells of the mesh structure 110 and decreases the possibility for the clot to dislodge from the mesh structure during extraction. The tight weave backstop 110a would catch the clot and pull it along.

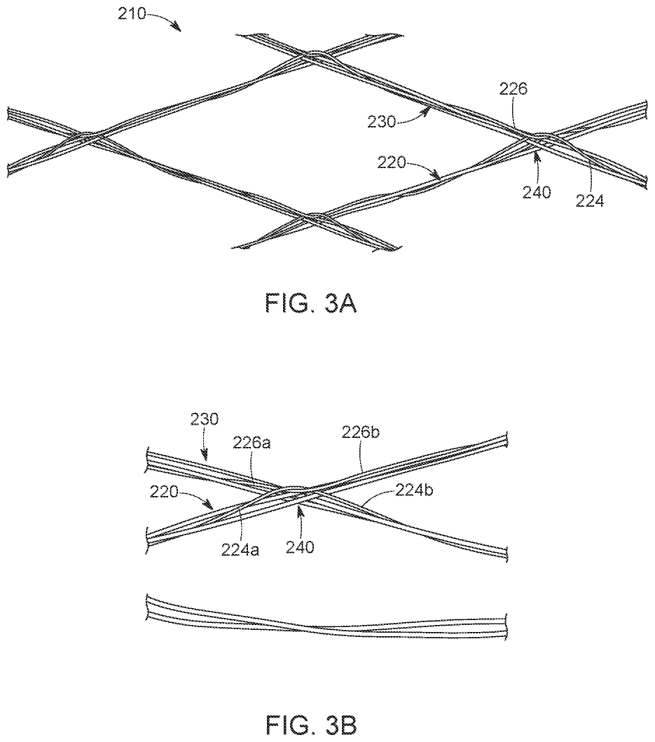

With reference to FIGS. 3A-3B, an example radially expandable body 210 according to alternative embodiments of the disclosure is shown. The radially expandable body 210 comprises plural sets of first filaments 220 extending spirally in a first direction from a proximal end portion to a distal end portion of the expandable body 210, and plural sets of second filaments 230 extending spirally in a second direction from the proximal end portion to the distal end portion. The plural sets of first filaments 220 and the plural sets of second filaments 230 may be constructed of a non-radiopaque material (non-radiopaque filaments). The plural sets of first filaments 220 intersect the plural sets of second filaments 230, forming plural cross sections 240 and defining plural openings of the radially expandable body 210. According to embodiments of the disclosure, the radially expandable body 210 may further include plural filaments constructed of a radiopaque material or radiopaque filaments 224, 226. At a cross section 240 where a set of first filaments 220 and a set of second filaments 230 intersect, a radiopaque filament 224 may twist with another radiopaque filament 226 and switch directions. For example, a first radiopaque filament 224 may include a first section 224a grouped with a set of first filaments 220 (spiraling in a first direction). A second radiopaque filament 226 may include a first section 226a grouped with a set of second filaments 230 (spiraling in a second direction). At a cross section 240 where a set of first filaments 220 and a set of second filaments 230 intersect, the first radiopaque filament 224 and the second radiopaque filament 226 may twist and switch directions. As such, a second section 224b of the first radiopaque filament 224 may be grouped with the set of second filaments 230 and a second section 226b of the second radiopaque filament 226 may be grouped with the set of first filaments 220.

With reference to FIGS. 3A-3B, in some embodiments, the non-radiopaque filaments in a set of first filaments 220 and in a set of second filaments 230 may be twisted. A radiopaque filament 224 may pass between the twisted non-radiopaque filaments 220, and/or, be twisted with the non-radiopaque filaments 230. In some embodiments, at a cross section where a set of first filaments and a set of second filaments intersect, one filament of a set of first filaments may pass between two filaments of a set of second filaments, and one filament of a set of second filaments may pass between two filaments of a set of first filaments. As such, the set of first filaments may rotate about a filament of the set of second filaments, and/or, the set of second filaments may rotate about a filament of the set of first filaments, thereby allowing the mesh body to freely expand to variable diameters. In alternative embodiments, it is not required that a filament of a set of first filaments pass between two filaments of a set of second filaments, or a filament of a set of second filaments pass between two filaments of a set of first filaments. At a cross section where a set of first filaments and a set of second filaments intersect, a radiopaque filament may twist with another radiopaque filament, locking or holding the intersection in place, creating a predetermined cell size of the overall mesh or expandable body.

In a non-limiting specific embodiment, the number of radiopaque filaments in the radially expandable body 210 may be the same as the total number of the sets of first and second filaments. By way of example, the radially expandable body may include totally six sets of filaments (three sets of first filaments and three sets of second filaments) and six radiopaque filaments. In alternative embodiments, the number of radiopaque filaments in the radially expandable body may be different from the total number of the sets of first and second filaments.

Intraluminal Device Including Laser-Cut Ribbons

Embodiments of the disclosure provide an intraluminal device which allows the user to control radial expansion and contraction of a mesh-like structure. The mesh-like structure can be formed by cutting a single tube of a material, optionally in combination of other wires or filaments. The cutting of a single tube can create an expandable body including plural elongate ribbons, either in a straight or spiral form, extending between the proximal end and distal end of the expandable body. The elasticity properties of a shape memory material, such as a nickel-titanium alloy, allow the expandable body to be properly shaped and radially evenly expanded. The ribbons created by cutting a tube may be provided with holes, slits, cuts, or other attachment features to allow additional wires or filaments to be threaded or looped through forming a mesh-like structure. The mesh-like structure can be expanded and/or contracted with a control member such as a pull wire. The pull wire allows the user to control the radial force of the mesh structure, facilitating penetration and removal of a clot from a blood vessel. Conventional stent retrievers consist of a structure made of a laser-cut tube that is expandable to a certain maximal diameter based solely on the shape memory property of the material, but lack the ability of control of the device integration into blood clots.

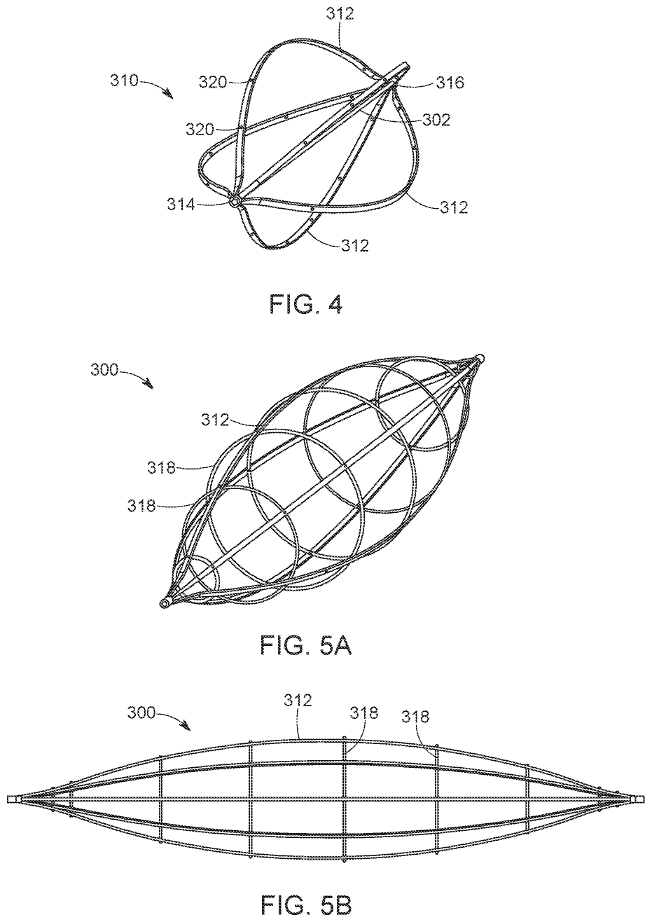

With reference to FIG. 4-5B, an example intraluminal device 300 of the disclosure in general comprises a radially expandable body 310 and a control member 302 operable to control radial expansion and contraction of the radially expandable body 310. The radially expandable body 310 comprises plural elongate ribbons 312 each extending from the proximal end portion 314 to the distal end portion 316 of the radially expandable body 310. One or more filaments 318 may be coupled to the plural elongate ribbons 312 forming a mesh structure.

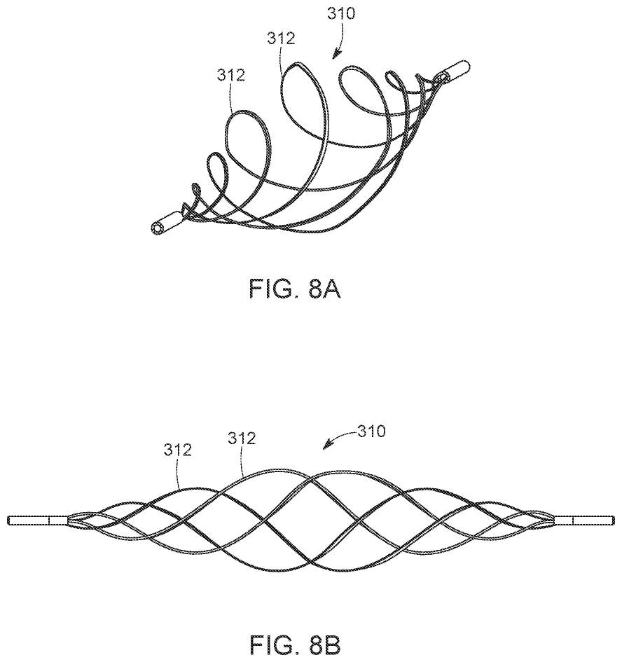

The plural elongate ribbons 312 may be formed by cutting a material from a single tube. By way of example, a pattern of plural slits may be laser-cut in a tubular member. The slits may be cut such that the plural ribbons 312 formed are generally in a straight or one-dimensional form (FIGS. 4, 5A-5B, and 6-7). The plural ribbons 312 may be evenly spaced apart circumferentially. Alternatively, slits may be cut from a single tube such that the plural ribbons 312 formed are in a spiral form extending from the proximal end to the distal end of the expandable body (FIGS. 8A-8B).

The tube or tubular member used for creating elongate ribbons 312 can be constructed of a shape memory material such as a metal alloy comprising nickel (Ni) and titanium (Ti), also known as Nitinol. Nitinol shape memory alloys can be deformed at a low temperature and are able to recover their original, permanent shape when exposed to a high temperature. Tubular members constructed of other shape memory materials can also be used to create elongate ribbons of a radially expandable body.

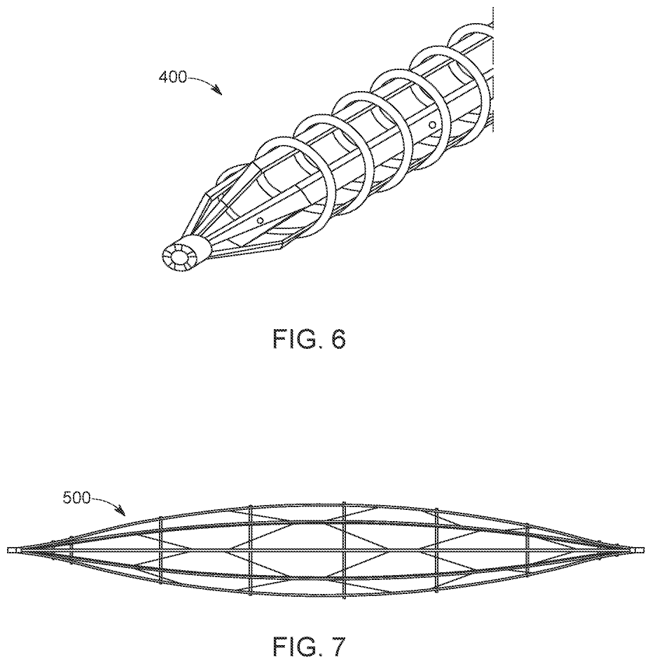

With reference to FIG. 4, attachment features such as holes, slits, cuts, or other features 320 can be created in the elongate ribbons 312 to allow wires or filaments 318 to be threaded or looped through to create a mesh-like structure. With reference to FIGS. 5A-5B, the attachment features 320 may be arranged to allow the filament or filaments 318 attached to the elongate ribbons 312 to form a circular configuration generally perpendicular to a longitudinal axis of the radially expandable body 310. Each of the plural circular filaments 318 intersects the plural elongate ribbons 312, forming a mesh-like structure of the intraluminal device 300. In an example intraluminal device 400 shown in FIG. 6, the attachment features may be arranged to allow the filament or filaments attached to the elongate ribbons to form a spiral configuration, extending spirally along the longitudinal axis of the radially expandable body. The spiraling filament or filaments intersect the plural elongate ribbons, forming a radially expandable mesh-like structure. In another example intraluminal device 500 shown in FIG. 7, the attachment features may be arranged to allow the filament or filaments to sway between two or more elongate ribbons in a sine-wave manner. The wavy filament or filaments intersect the plural elongate ribbons, forming a radially expandable mesh-like structure.

FIGS. 8A-8B illustrate an alternative embodiment of the disclosure, wherein plural elongate ribbons 312 are formed in a spiral shape. The elongate ribbons 312 in a spiral shape can be formed by cutting a material from a single tube in a one-way direction, clockwise or counterclockwise. Attachment features such as holes, slits, cuts, or other features can be created in the elongate ribbons 312 to allow one or more filaments to be coupled to the elongate ribbons. The attachment features may be arranged to allow the filament or filaments attached to the elongate ribbons 312 to form a spiral configuration, extending spirally along the longitudinal axis of the radially expandable body. The filament or filaments may extend in a spiral direction opposite to the spiral direction of the elongate ribbons 312, forming a mesh-like structure of the intraluminal device.

Various embodiments of an intraluminal device have been described. Advantageously, the intraluminal device of the disclosure allows the user to control the outer diameter of a mesh structure, and pulse or manipulate radial force of the mesh structure to facilitate penetration and retrieval of blood clots. The pattern of filament cross sections allows the mesh structure to expand to variable diameters while maintaining a predetermined cell size. This helps create a precise repeatable expansion and radial force. Free and precise expansion allows for ease of application of radial force and creates tactile feedback to the user. In conventional stent retrievers, the cross sections of filaments slip during expansion, causing the size of one cell to be reduced and the size of another cell to be increased. In some conventional stent retrievers, filaments at cross sections weave too tightly into each other, limiting a free expansion state and creating inconsistent performance. The intraluminal device of the disclosure includes radiopaque filament or filaments to assist in holding the cell structure and creating radiopacity for the mesh structure. The intraluminal device of the disclosure also allows for a large cell structure while still maintaining structural integrity, which in turn allows for holding a larger majority of a blood clot together. Keeping a larger majority of the clot intact ensures that the entire clot can be removed the first time and small pieces do not detach. The intraluminal device allows for a distal portion with a tighter weave pattern serving as a backstop when removing a clot. A tight weave pattern creates smaller cells and decreases the possibility for a clot to dislodge from the mesh structure during extraction. If a clot does slip, the tight weave backstop can catch the clot and pull it along.

In use, an intraluminal device of the disclosure may be delivered to a target site using a microcatheter. A microcatheter may be first introduced to the target site through an access in the patient using a guiding catheter. The microcatheter may be guided to the target site through the use of a guidewire. The guidewire may be visible via fluoroscopy, allowing the microcatheter to be reliably advanced over the guidewire to the target site. Once the target site has been accessed with the microcatheter tip, the guidewire can be withdrawn, clearing the lumen of the microcatheter. The intraluminal device of the disclosure can be placed into the proximal open end of the microcatheter and advanced through the microcatheter. In delivery to the target site, the intraluminal device can be in a thin compact form to allow the device to penetrate through e.g., a blood clot at the target site. By using an operating device such as handle, the user may pulse or manipulate the radial force of the mesh structure to aid the device to travel through the clot. Once the mesh body or at least the distal portion of the mesh body has travelled through and is incapsulated in the clot, the user can expand the device. The user may control the radial expansion of the device, for example, expand the device more for a larger vessel or less for a smaller vessel. During expansion, the mesh structure penetrates radially through the clot and creates a hold on the bulk of the clot. The user can then remove the clot from the vessel by retrieving the intraluminal device.

Various embodiments of an intraluminal device have been described with reference to figures. It should be noted that the figures are intended to facilitate illustration and some figures are not necessarily drawn to scale. Further, in the figures and description, specific details may be set forth in order to provide a thorough understanding of the disclosure. It will be apparent to one of ordinary skill in the art that some of these specific details may not be employed to practice embodiments of the disclosure. In other instances, well known components or process steps may not be shown or described in detail in order to avoid unnecessarily obscuring embodiments of the disclosure.

All technical and scientific terms used herein have the meaning as commonly understood by one of ordinary skill in the art unless specifically defined otherwise. As used in the description and appended claims, the singular forms of “a,” “an,” and “the” include plural references unless the context clearly dictates otherwise. The term “or” refers to a nonexclusive “or” unless the context clearly dictates otherwise. The term “proximal” and its grammatically equivalent refers to a position, direction or orientation towards the user or physician's side. The term “distal” and its grammatically equivalent refers to a position, direction or orientation away from the user or physician's side. The term “first” or “second” etc. may be used to distinguish one element from another in describing various similar elements. It should be noted the terms “first” and “second” as used herein include references to two or more than two. Further, the use of the term “first” or “second” should not be construed as in any particular order unless the context clearly dictates otherwise.

Those skilled in the art will appreciate that various other modifications may be made. All these or other variations and modifications are contemplated by the inventors and within the scope of the invention.

What is claimed is:

1. An intraluminal device comprising a radially expandable body having a proximal end portion and a distal end portion, and a control member operable to expand and contract the radially expandable body,
wherein the radially expandable body comprises plural sets of first filaments spiraling in a first direction from the proximal end portion to the distal end portion and plural sets of second filaments spiraling in a second direction from the proximal end portion to the distal end portion, the plural sets of first filaments intersecting the plural sets of second filaments, forming plural cross sections of the radially expandable body; and
wherein at least one of the plural sets of first filaments comprises a filament constructed of a radiopaque material.

2. The intraluminal device of claim 2, wherein each of the plural sets of first filaments comprises a filament constructed of a radiopaque material.

3. The intraluminal device of claim 2, wherein each of the plural sets of second filaments is free of a filament constructed of a radiopaque material.

4. The intraluminal device of claim 1, wherein each of the plural sets of first filaments comprises two filaments that are twisted and each of the plural sets of the second filaments comprises two filaments that are twisted.

5. The intraluminal device of claim 4, wherein at a cross section where one of the plural sets of first filaments and one of the plural sets of second filaments intersect, one of the two filaments of the one of plural sets of first filaments passes between the two filaments of the one of plural sets of second filaments, and one of the two filaments of the one of plural sets of second filaments passes between the two filaments of the one of plural sets of first filaments.

6. The intraluminal device of claim 5, wherein the plural sets of first filaments each comprises a radiopaque filament and a non-radiopaque filament, and the plural sets of second filaments each comprises two non-radiopaque filaments.

7. The intraluminal device of claim 1, wherein the control member comprises a pull wire having a distal end portion coupled to the distal end portion of the radially expandable body, allowing to apply an axial force to move the distal and proximal end portions of the radially expandable body relative to each other.

8. An intraluminal device comprising a radially expandable body having a proximal end portion and a distal end portion, and a control member operable to expand and contract the radially expandable body, wherein the radially expandable body comprises:
plural sets of first filaments spiraling in a first direction from the proximal end portion to the distal end portion;
plural sets of second filaments spiraling in a second direction from the proximal end portion to the distal end portion, the plural sets of second filaments intersecting the plural sets of first filaments, forming plural cross sections of the radially expandable body; and
plural radiopaque filaments comprising at least a first radiopaque filament and a second radiopaque filament, wherein the first radiopaque filament comprises a first section grouped with one of the plural sets of first filaments, the second radiopaque filament comprises a first section grouped with one of the plural sets of second filaments, and wherein the first radiopaque filament and the second radiopaque filament are twisted at a cross section where the one of the plural sets of first filaments and the one of the plural sets of second filaments intersect, to allow a second section of the first radiopaque filament to be grouped with the one of the plural sets of second filaments and allow a second section of the second radiopaque filament to be grouped with the one of the plural sets of first filaments.

9. The intraluminal device of claim 8, wherein each of the plural sets of first filaments comprises two filaments that are twisted, and each of the plural sets of second filaments comprises two filaments that are twisted.

10. The intraluminal device of claim 9, wherein a number of the plural radiopaque filaments is equal to a total number of sets of first and second filaments.

11. An intraluminal device, comprising a radially expandable body having a proximal end portion and a distal end portion and a control member operable to expand and contract the radially expandable body, wherein the radially expandable body comprises:
plural elongate ribbons each extending from the proximal end portion to the distal end portion; and
one or more filaments coupled to the plural elongate ribbons forming a mesh structure.

12. The intraluminal device of claim 11, wherein the plural elongate ribbons are formed by cutting plural slits in a tubular member.

13. The intraluminal device of claim 11, wherein each of the plural ribbons comprises plural attachment features to couple the one or more filaments.

14. The intraluminal device of claim 11, wherein the plural attachment features are arranged to allow the one or more filaments to form plural circular filaments each intersecting the plural elongate ribbons, the plural circular filaments being generally perpendicular to a longitudinal axis of the radially expandable body.

15. The intraluminal device of claim 11, wherein the plural attachment features are arranged to allow the one or more filaments to extend spirally from the proximal end portion to the distal end portion and intersect the plural elongate ribbons.

16. The intraluminal device of claim 11, wherein the plural attachment features are arranged to allow the one or more filaments to sway between two or more elongate ribbons in a sine-wave manner.

17. The intraluminal device of claim 11, wherein the plural elongate ribbons are constructed of a shape memory material.

18. The intraluminal device of claim 11, wherein the plural elongate ribbons are generally evenly spaced apart circumferentially.

19. The intraluminal device of claim 11, wherein the plural elongate ribbons extend spirally from the proximal end portion to the distal end portion in a first direction, and the one or more plural filaments extend spirally from the proximal end portion to the distal end portion in a second direction opposite to the first direction.

20. The intraluminal device of claim 19, wherein the plural elongate ribbons are formed by cutting plural slits in a spiral pattern in a tubular member constructed of a shape memory material.
